(19) United States
(12) Patent Application Publication
(10) Pub. No.: US 2011/0281294 A1
(43) Pub. Date: Nov. 17, 2011

(54) METHOD AND DEVICE FOR MEASURING ADHESION FORCES

(75) Inventors: Marko Pudas, Oulu (FI); Markus Polet, Vantaa (FI); Risto Sipola, Oulu (FI)

(73) Assignee: NANOGALAX LTD OY, Piispanristi (FI)

(21) Appl. No.: 13/130,639

(22) PCT Filed: Nov. 18, 2009
(86) PCT No.: PCT/FI2009/050932

§ 371 Date: Jul. 20, 2011

(30) Foreign Application Priority Data
Nov. 21, 2008 (FI) 20086109

(51) Int. Cl. C12Q 1/02; G01N 19/04; C12M 1/34

(52) U.S. Cl. 435/29; 435/287.1; 73/150 A

(57) ABSTRACT

A measurement head for measuring the adhesion force between a surface and a particle attached to the surface, a force measurement system and a method for measuring the adhesion force between a surface and a particle attached to the surface are described.

### FIELD OF THE INVENTION

The present invention relates to a measurement head for measuring the adhesion force between a surface and a particle, such as a cell, attached to said surface. The present invention also relates to a force measurement system and to a method for measuring the adhesion force between a surface and a particle attached to said surface.

### BACKGROUND OF THE INVENTION

In some fields of research it is necessary to define how tightly a particle is adhered to a surface or to each other. One example is cell culturing wherein the cells may be grown as a layer on a surface, such as a culture plate. Generally such adhesion has been defined for example by adding a degrading enzyme to the cell culture and monitoring how long it takes until the cells are detached from the surface.

U.S. Pat. No. 4,831,869 discloses a method and device for measuring the adhesion of cells to a substratum wherein a flow cell is provided and a fluid comprising a suspension of the cells is accelerated and the flow rates are measured. With such methods and devices it is virtually impossible to accurately measure the adhesion of one specific particle, such as a cell. Therefore there is a need for more sophisticated methods and devices for measuring the adhesion of particles to a surface.

Adhesion of a single cell to a substrate is mediated by transmembrane molecules, i.e. integrin binding to extracellular matrix (ECM). The single integrin binding force is translated into intracellular structures and cells form specific structures to organize specific binding sites, usually at the cell periphery. It has been previously shown that the adhesion force of a single integrin molecule to ECM is ˜50 nN. The total number of integrins in cell attachment varies from 10 000 to 1 000 000 single molecules. So far real measurements of total living cell attachment force have not been recorded due to lack of suitable equipment, which should have sufficient measuring range combined with nano scale resolution.

The actual adhesion force of a single living cell to the ECM gives unique information of the properties of the substrate, cell culture media and environmental effects, such as the impact of light and temperature, and also of the organization of the cell as such. Furthermore, different cells, such as malignantly transformed cells, are known to have different binding affinities to various substrates and this binding can be modulated by for example anti-cancer medication.

The present inventors have built an instrument for studying the total cell-ECM binding force. By a specific mechanical arrangement a whole cell can be detached intact from ECM under a phase contrast light microscope. By carrying out the pull-out sequence in single action it is possible to analyze the total cell-ECM binding force and additionally to associate podosome/binding structure forces. The process of cell pull-out can be varied and last as long as for one second, which is thought to enable real receptor-ligand dissociation and changes in receptor conformation.

As an improvement to any simple AFM based studies, this total cell-adhesion measurement method allows relating the cellular structures and changes within these macromolecules to real, reproducibly recordable forces. The preliminary results obtained with this novel instrumentation and calibrated estimates of the total cell-ECM binding force and the number of integrins required for this force are presented herein. The present inventors have found a high variation in the adhesion of different cells; hence the equipment has a large dynamic range. This is achieved by attaching the instrument to a computer connected to a 3D positioner, to guide the 3D actuator. The latest development of the linear piezo positioners and microcontroller electronics have made it possible to achieve a resolution of <20 nm with movements up to 40 mm, with force detection <1 μN and response speed of ˜10 ms.

### BRIEF DESCRIPTION OF THE INVENTION

The object of the invention is presented in the independent claims. Some preferred embodiments are presented in the dependent claims.

One aspect of the present invention relates to a measurement head for measuring the adhesion force between a surface and a particle attached to said surface, wherein the measurement head comprises
a sharp edge part adapted to be introduced (mounted) at least partially into the particle when the measurement head is introduced, such as pushed, against the particle essentially in the direction of the plane of the surface or up from said surface in an angle, and
a support part having a width essentially in the magnitude of the particle dimension and which is adapted to limit the mounting of the sharp edge part interacting with the particle and to allocate a force to the body of the particle essentially in the direction of the plane of the surface or up from said surface in an angle.

Another aspect of the present invention relates to a force measurement system, which is arranged to measure the forces allocated to the measurement head of the invention.

Still another aspect of the present invention relates to a method for measuring the adhesion force between a surface and a particle attached to said surface, wherein the measurement head of the invention is introduced (mounted) against the particle, and introduced, such as pushed, against said particle essentially to the direction of the plane of the surface or up from said surface in an angle, and the forces allocated to the measurement head are measured to determine said adhesion force.

Still another aspect of the present invention relates to a research device for measuring the adhesion force between a surface and a particle attached to said surface.

Still another aspect of the present invention relates to a control system arranged to control the measurement head of the invention.

Still another aspect of the present invention relates to a computer readable data storage medium having computer-executable program code stored which is operative to perform a method of the invention when executed on a computer.

It is an advantage of the present invention that a single particle, such as a cell, can be picked up specifically. Furthermore, if a cell or the like is picked, it will maintain its form and will not easily break or get squashed. This way the specific adhesion force between a single particle and the surface can be measured accurately.

### BRIEF DESCRIPTION OF THE DRAWINGS

FIG. 1 shows the form of one embodiment of the measurement head comprising a needle-like structure wherein the tip of the needle forms the sharp edge part and a part of the needle arm bent perpendicularly to said tip forms the support part.

FIG. 2A shows how the measurement head is approaching a cell adhered to a surface. The arrows A and B show two optional movement directions for the measurement head after it has been mounted to the cell.

FIGS. 2B-D show exemplary positions of the sharp-edge part of the measurement head according to exemplary embodiments of the present invention.

FIG. 3 shows a block diagram of an exemplary cell adhesion measurement system.

FIG. 4 shows how the force begins to increase when the measurement head grabs the cell.

### DETAILED DESCRIPTION OF THE INVENTION

The present invention provides a measurement head, system and a method for measuring the adhesion force between a surface and a particle attached to said surface. Said surface may be any suitable surface the particle may be adhered to. The particle may be for example a cultured cell, a metal particle or a polymer particle. Examples of such cells include fibroblasts and stem cells.

The measurement head comprises a sharp edge part adapted to be introduced, such as mounted at least partially into the particle when the measurement head is introduced, such as pushed, against the particle essentially in the direction of the plane of the surface or up from said surface in an angle. If the particle contains a wall and optionally other inner compartments, i.e. the particle of a cell or the like, the sharp edge part may penetrate the wall. Generally the length of the sharp edge part is at the range of 0.1-50 μm, preferably 10-15 μm.

The measurement head also comprises a support part having a width essentially in the magnitude of the particle dimension and which is adapted to limit the mounting of the sharp edge part into the particle and to allocate a force to the body of the particle essentially in the direction of the plane of the surface or up from said surface in an angle. Therefore, according to an exemplary embodiment, the sharp edge part does not completely penetrate through the particle, but only to a certain limit. According to an embodiment of the invention the penetration can even be prevented. Generally, according to an exemplary embodiment of the invention, the width of the support part is at the range of 1-500 μm, preferably 30-40 μm.

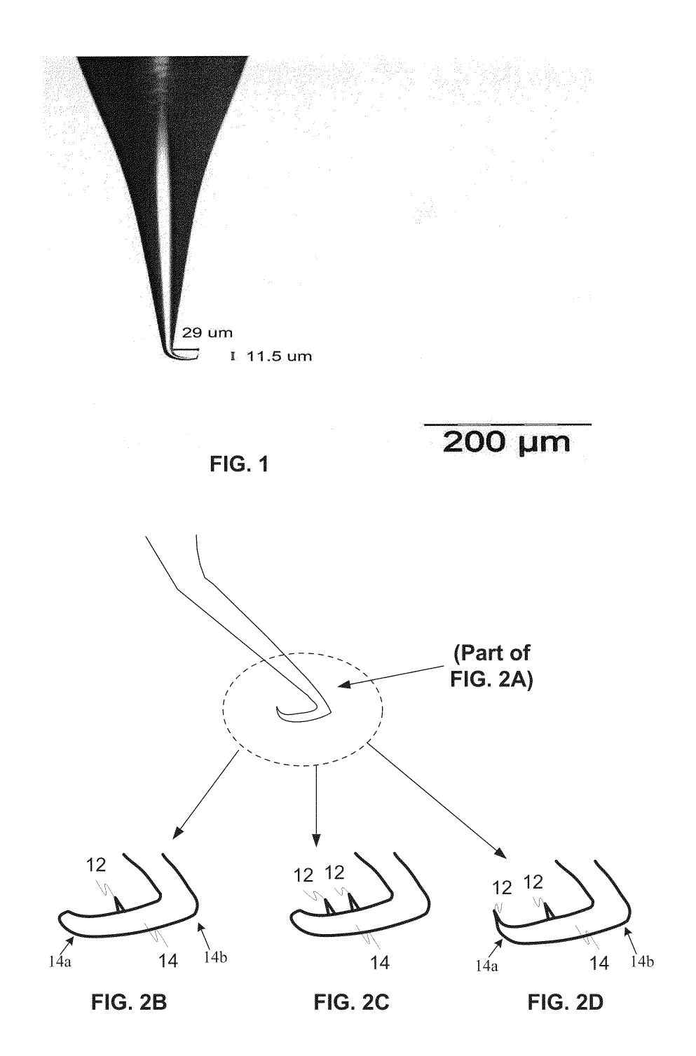

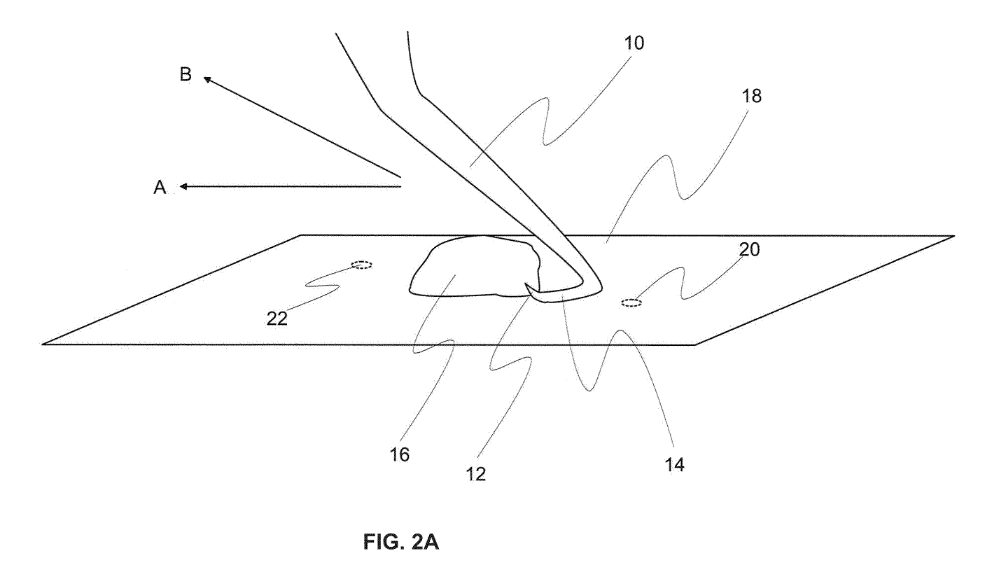

In one embodiment the measurement head comprises a needle-like structure wherein the tip of the needle forms the sharp edge part and a part of the needle arm bent essentially perpendicularly to said tip forms the support part. Generally the rest of the needle arm continues essentially perpendicularly from the support part therefore forming a J-shaped structure (FIGS. 1 and 2). Such a needle may be prepared from a metal wire, such as tungsten wire, and sharpened by methods known in the art, such as by reverse electrolysis.

In another embodiment the measurement head comprises a spiral-like needle structure wherein the tip of the needle forms the sharp edge part and wherein the rise of the spiral is adjusted to maintain the spiral at the level of the particle during a half of the first round of the spiral.

Said measurement head can also be utilized as a grabbing or picking means for picking up a specific particle. For example one cell can be picked up and moved to another location for further processing and/or examination.

The present invention also provides a method for measuring the adhesion force between a surface and a particle attached to said surface, wherein the measurement head of the invention is mounted against the particle, pushed against said particle in the direction of the plane of the surface or up from said surface in an angle, and the forces allocated to the measurement head are measured to determine said adhesion force.

In one embodiment the measurement head is pushed and moved against said particle substantially in the direction of the plane of the surface (arrow A in FIG. 2). In another embodiment the measurement head is pushed and moved against said particle up from said surface in an angle (arrow B in FIG. 2). Said angle may be in the range of 1-90°. The measurement head may also be moved by combining the above-mentioned embodiments, e.g. measurement head is first pushed and moved against said particle substantially in the direction of the plane of the surface and then up from said surface in an angle.

In one embodiment of the present invention the starting point and the end point for the measurement are defined before the measurement. This may be carried out by using the measurement head.

FIG. 2A illustrates the measurement operation with the needle-like measurement head of the invention. The needle-like measurement head (10) having the sharp edge part (12) and the support part (14) is approaching a cell (16) adhered to a surface (18). The starting point (20) and the end point (22) have been defined at the both sides of the cell. When the measurement head is mounted to the cell, it can be moved either in the direction of the plane of the surface (A) or up from said surface in an angle (B). Preferably in the measurement situation the sharp edge part (12) points to at least some degree up from the plane of the surface.

The present invention also provides a force measurement system or device comprising means for measuring, which is arranged to measure the forces allocated to the measurement head of the invention. Generally said system is arranged to move said measurement head (i.e. the device may be called a robot), preferably to perform the method of the invention. The measurement head is attached to a probe which senses the forces allocated to the measurement head, and the probe is connected to a central data processing and storage system for collecting, storing and processing the gathered force data. Finally the adhesion forces are defined and outputted.

The measurement head may be moved manually, automatically or partly by both ways. For example a user may manually set up the starting point and the end point for the measurement, direct the measurement head to the particle, and the automated system may continue from that point moving the head and simultaneously measuring and recording the forces.

Such force measurement system platform may contain several devices for use in the measurement, such as 3D controller, actuator, probe, control unit, storage media, output device such as a display or printer, other electronic, connectors and the like.

A haptic 3D controller (user control interface) gives the user manual control over the robot. The controller advantageously comprises a pen-like handle that can be moved with e.g. six degrees of freedom. The “pen” may have buttons that can be assigned to different functions. The controller may also provide a haptic feedback option that can be used to let the user feel what the robot feels. This is a useful feature when the user wants to manually manipulate the particle or just feel the surroundings of the probe.

A linear piezo actuator device comprises a control device and three linear piezo stages with position encoders. The controller may be connected to a control unit, such as a PC, for example with a USB connector. The controller software can feed the control device commands to move the actuators. The linear actuators can move with a positioning resolution of 5 nm. Motion is set by either relative increments to a position or by absolute coordinates. In one example the range of each actuator is 4 cm. The actuator stack enables the robot to operate in a 4 cm×4 cm×4 cm space. In one example the robot software runs on a regular PC with a Windows XP operating system.

The control unit may be arranged to perform several tasks. These include e.g. receiving user input from 3D controller, controlling the actuator and moving the measurement head, collecting, storing and processing the data obtained from the force probe and/or the camera, and defining and outputting the adhesion force between a surface and a particle attached to said surface. The control unit may read a computer readable data storage medium having computer-executable program code stored which is operative to perform a method of the invention when executed on a computer i.e. to perform one or more of the above-mentioned tasks by using the required devices.

The probe can be customized for every device separately. In one test case the probe was a silicon strain-gauge force sensor, which was attached to a mechanically custom-designed arm. The probe was connected to a voltage amplifier, which again was connected to a 24-bit precision AD converter. The measurement head of the present invention is then attached to the force sensor to feel the surroundings.

It is to be noted that FIG. 2A shows only an exemplary measurement head (10) and that the sharp edge part (12) may also locate in different positions relating to the support part (14) than shown in FIG. 2A (where the sharp edge part locates in the end of the support part (14), i.e. the tip of the support part forms the sharp edge part). FIGS. 2B-2D show other exemplary positions of the sharp edge part (12) of the measurement head according to exemplary embodiments of the present invention, where in FIG. 2B the sharp edge part (12) locates between the tip (14a) and root (14b) of the support part (14) of the measurement head (10).

Furthermore, according to an embodiment of the invention the measurement head (10) may comprise also additional sharp edge parts, such as the sharp edge parts (12) locating on the support part (14) illustrated in FIG. 2C. In addition FIG. 2D shows another exemplary, where the tip (14a) of the support part (14) forms the sharp edge part (12) (similarly as in FIG. 2A) and in addition the additional sharp edge part is arranged between the tip (14a) and root (14b) of the support part (14) of the measurement head (10).

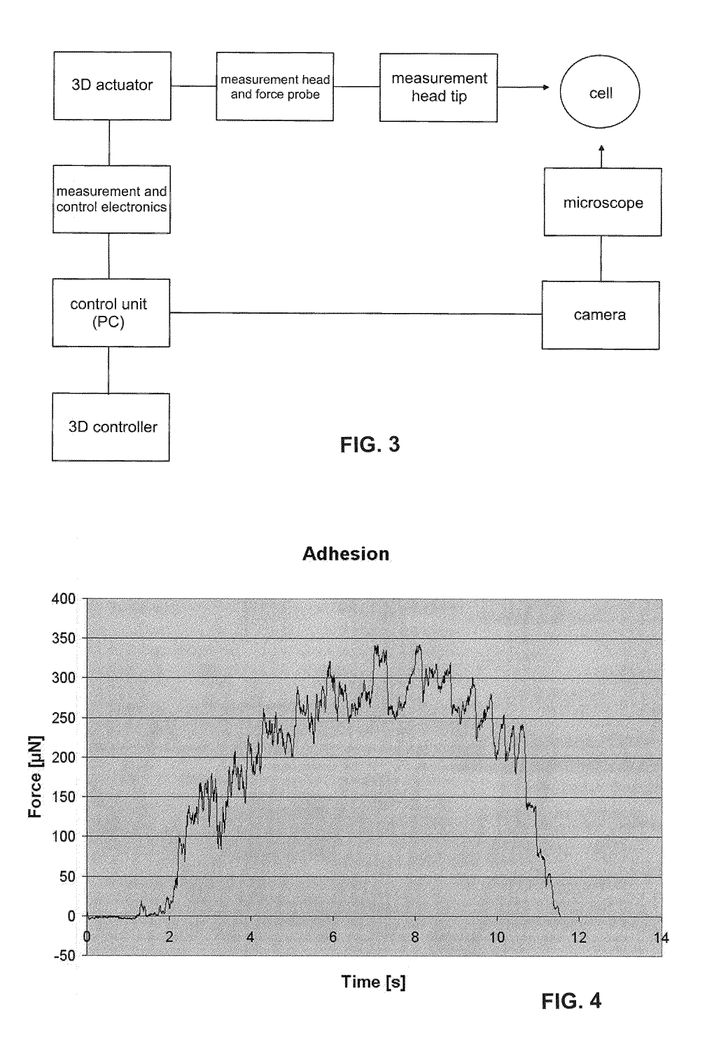

In one embodiment the force measurement system is a cell adhesion measurement system. FIG. 3 shows a block diagram of an exemplary set up of said system. A control unit (which is a PC in this case and may be implemented at least partially by a computer program code means), controls the measurement and control electronics and therefore the movement of the measurement head. Also a camera is connected to the control unit for monitoring the microscope view. The user may operate the 3D actuator with the 3D controller also connected to the control unit. The control unit also collects, stores and processes the data from the force probe.

### EXAMPLES

The next example illustrates how the measurement head may be moved in the adhesion measurement. The scenario is the following.

The robot will remove a particle placed on a substrate (surface). The removal will be done by sweeping the measurement head's tip over the particle. During this movement the tip is not allowed to touch the surface of the substrate. Touching the surface would make the measurement invalid. The scraping movement's starting and ending points are programmed by a human user. The user moves the actuator with the haptic 3D controller and marks these points by simply making the tip of the measurement head touch the substrate surface at desired points. The particle should of course be located between these points. After both points are marked the robot automatically performs the scraping motion and removes the particle. The adhesion force of the particle is measured during the removal.

The scenario can be divided into five stages.

Stage 1: Marking the measurement ending point. The user steers the actuator with a 3D controller. The measurement head is moved across the substrate above a point where the actuator should stop the measurement. The measurement head is then pressed against the substrate, causing the force sensor to react. When the force signal rises above a threshold value, the next stage is triggered to start.

Stage 2: The robot automatically searches the surface. The goal is to lift the tip of the measurement head slightly above the substrate (less than 1 μm) and mark this point as the measurement ending point. The challenge in this stage is to cope with the noise of the force sensor, drift and inaccurate calibration. Because of the uncertain conditions, the tip bend can not be calculated straight from the initial force signal value that triggered this stage. The nanoscale actuator can move very accurately and this is the quality we take advantage of. The sensor can be used e.g. to tell the robot if there is a contact with the surface or not. The objective is not to estimate how far the surface is or how much the tip is bent according to the sensor. A method that in some way resembles the way of approximating the zero of a function is used. The robot (iteratively) seeks the boundary of the surface by driving the actuator up and down. When the status of the contact changes, the robot changes the direction of the motion and cuts the distance it drives in half. By repeating this behavior the tip closes to the surface boundary. This way the robot can achieve the desired distance from the surface quickly. The distance remains inaccurate, but still there is certainty that the tip is somewhere within an appropriate space. After finding the surface and “picking-up” the ending point, the robot gives control back to the human user and moves on to the next stage.

Stage 3: Marking the measurement starting point. This stage is similar to the first one, except that this time the user moves the measurement head above the desired starting point of the measurement. Again, as the force exceeds the threshold value, the next stage is triggered.

Stage 4: Searching the surface. This stage is similar to the second one, except this time after finding the measurement starting point the robot begins the particle removal stage.

Stage 5: Removal of the particle and adhesion measurement. The robot starts to move the measurement head from the starting point to the ending point along a straight-line trajectory. The tip travels over the surface without touching it. As the tip travels it touches the particle on its way and the same sensor that was earlier used to detect the surface is now used to measure the adhesion force of the particle. During the removal more than one module controls the actuator trajectory. One module keeps the actuator approaching the goal point (ending point) while another module is in charge of keeping the actuator in a straight line. In addition there are also reflexes present, but they do not take action if the removal is successful.

FIG. 4 is an exemplary graph of the measured adhesion forces. It can be seen that when the measurement head grabs the cell, the force begins to increase. It is believed that the peaks are caused by the tightening and loosening of the cell adhesions. The force reaches highest level in the middle of the sweep when several cell adhesions are stressed. The force decreases when the adhesions are loosen from the surface and finally reach the zero level when the cell has come loose.

1. A measurement head for measuring the adhesion force between a surface and a particle attached to said surface, characterized in that the measurement head comprises
at least one sharp edge part adapted to be introduced, such as mounted, at least partially with the particle when the measurement head is pushed against the particle in the direction of the plane of the surface or up from said surface in an angle, and
a support part having a width essentially in the magnitude of the particle dimension and which is adapted to limit the mounting of the sharp edge part interacting with the particle and to allocate a force to the body of the particle in the direction of the plane of the surface or up from said surface in an angle.

2. The measurement head of claim 1, characterized in that it comprises a needle-like structure and a part of the needle arm bent essentially perpendicularly to the tip forms the support part.

3. The measurement head of claim 1, characterized in that it comprises a spiral-like needle structure and wherein the rise of the spiral is adjusted to maintain the spiral at the level of the particle during a half of the first round of the spiral.

4. The measurement head of claim 1, characterized in that the tip of the needle forms the sharp edge part and/or wherein the sharp edge part is arranged into the support part.

5. The measurement head of claim 1, characterized in that the length of the sharp edge part is at the range of 0.1-50 μm, preferably 10-15 μm, and/or the width of the support part is at the range of 1-500 μm, preferably 30-40 μm.

6. A method for measuring the adhesion force between a surface and a particle attached to said surface, characterized in that the measurement head of claim 1 is introduced with the particle, pushed against said particle in the direction of the plane of the surface or up from said surface in an angle, and the forces allocated to the measurement head are measured to determine said adhesion force.

7. The method of claim 6, characterized in that before the measurement the starting point and the end point for the measurement are defined.

8. A force measurement system comprising means for measuring forces, characterized in that the system is arranged to measure the forces allocated to the measurement head of claim 1.

9. The force measurement system of claim 8, characterized in that the force measurement system is arranged to move said measurement head.

10. The force measurement system of claim 8, characterized in that the force measurement system is arranged to perform a method for measuring the adhesion force between a surface and a particle attached to said surface, wherein said measurement head is introduced with the particle, pushed against said particle in the direction of the plane of the surface or up from said surface in an angle, and the forces allocated to the measurement head are measured to determine said adhesion force.

11. The measurement head of claim 1, characterized in that the particle is a cell.

12. Computer program product comprising a computer-executable program code, which is adapted to perform the method steps of claim 6, when said computer-executable program code is executed on a computer.
